(19) United States
(12) Patent Application Publication
(10) Pub. No.: US 2015/0016486 A1
(43) Pub. Date: Jan. 15, 2015

(54) REMOTE ELECTROMIGRATION MONITORING OF ELECTRONIC CHIPS

(71) Applicant: International Business Machines Corporation, Armonk, NY (US)

(72) Inventors: Graeme A. Hutcheon, Essex Junction, VT (US); Baozhen Li, South Burlington, VT (US); K. Paul Muller, Wappingers Falls, NY (US)

(21) Appl. No.: 14/501,710

(22) Filed: Sep. 30, 2014

Related U.S. Application Data
(63) Continuation of application No. 13/804,657, filed on Mar. 14, 2013.

(51) Int. Cl. G01N 25/00; G01R 31/26; G01N 33/00

(52) U.S. Cl. CPC G01N 25/00; G01N 33/00; G01R 31/2642; G01N 2033/0095
USPC 374/45

(57) ABSTRACT

A method of remotely monitoring electromigration in an electronic chip includes sensing, at a first location, at least one temperature value of the electronic chip, sending the at least one temperature value to a remote monitoring system, accumulating a plurality of temperature values of the electronic chip at the monitoring system during a reporting period, calculating an Electromigration Life Consumed (EMLC) value of the electronic chip for the reporting period based on the plurality of temperature values, determining whether the EMLC of the electronic chip is above a predetermined threshold, and providing a signal when the EMLC of the electronic chip is above the predetermined threshold.

This application is a continuation of U.S. application Ser. No. 13/804,657 filed Mar. 14, 2013, the disclosure of which is incorporated by reference herein in its entirety.

### BACKGROUND

The present invention relates to the art of monitoring and more particularly to a system and method for remote electromigration monitoring of electronic chips.

Electromigration describes a phenomenon associated with current flow through a conductor. Current flowing through a conductor causes ions in the conductor to gradually move. Movement of the ions results from a momentum transfer between conducting electrons and diffusing metal ions. Electromigration is of particular interest in microelectronics. More specifically, electromigration increases as conductor size decreases. Over time, an effective life of microelectronic components, such as integrated circuit chips, processor chips, memory chips, will decrease and ultimately end as a result of electromigration. As a microelectronic component nears an end of its effective life, periodic glitches could occur resulting from electromigration. Over time, an overall number and duration of the periodic glitches could increase until the microelectronic chip ultimately fails.

Electronic chips are designed to a specific electromigration specification. A typical electromigration specification identifies an operational life at a particular threshold temperature. For example, an electronic chip may have an electromigration specification of 75° C., and 100 k Power On Hours (POH). Electromigration risk increases exponentially above a threshold temperature. Thus, operation above the threshold temperature would reduce the operational life. In contrast, operation below the threshold temperature may elongate the operational life.

### SUMMARY

According to one exemplary embodiment, a method of remotely monitoring electromigration in an electronic chip includes sensing, at a first location, at least one temperature value of the electronic chip, sending the at least one temperature value to a remote monitoring system, accumulating a plurality of temperature values of the electronic chip at the monitoring system during a reporting period, calculating an Electromigration Life Consumed (EMLC) value of the electronic chip for the reporting period based on the plurality of temperature values, determining whether the EMLC of the electronic chip is above a predetermined threshold, and providing a signal when the EMLC of the electronic chip is above the predetermined threshold.

In accordance with another exemplary embodiment, a computer program product includes a computer useable medium having a computer readable program. The computer readable program, when executed on a computer, causes the computer to sense, at a first location, at least one temperature value of the electronic chip, send the at least one temperature value to a remote monitoring system, accumulate a plurality of temperature values of the electronic chip at the monitoring system during a reporting period, calculate an Electromigration Life Consumed (EMLC) value of the electronic chip for the reporting period based on the plurality of temperature values, determine whether the EMLC of the electronic chip is above a predetermined threshold, and provide a signal when the EMLC of the electronic chip is above the predetermined threshold.

Additional features and advantages are realized through the techniques of the present invention. Other embodiments and aspects of the invention are described in detail herein and are considered a part of the claimed invention. For a better understanding of the invention with the advantages and the features, refer to the description and to the drawings.

### BRIEF DESCRIPTION OF THE SEVERAL VIEWS OF THE DRAWINGS

The subject matter which is regarded as the invention is particularly pointed out and distinctly claimed in the claims at the conclusion of the specification. The forgoing and other features, and advantages of the invention are apparent from the following detailed description taken in conjunction with the accompanying drawings in which:

FIG. 1 is a block diagram illustrating a remote Electromigration Monitoring system coupled to a plurality of electronic systems in accordance with an exemplary embodiment;

FIG. 2 is a block diagram illustrating a plurality of electronic chips of one of the plurality of electronic systems of FIG. 1;

FIG. 3 is a graphical depiction of a temperature profile for one of the plurality of chips of FIG. 2 for a first reporting period;

FIG. 4 is a graphical depiction of a temperature profile for one of the plurality of chips of FIG. 2 for a second reporting period;

FIG. 5 is a graphical depiction of a temperature profile for one of the plurality of chips of FIG. 2 for a third reporting period;

FIG. 6 is a graphical depiction of a temperature profile for one of the plurality of chips of FIG. 2 for a fourth reporting period;

FIG. 7 is a graphical representation of a high temperature of the electronic chip for each of the first, second, third and fourth reporting periods;

FIG. 8 is a flow diagram illustrating a method of monitoring Electromigration Life Consumed (EMLC) for the electronic chip of FIG. 2;

FIG. 9 is a block diagram illustrating an electronic chip including a plurality of cores coupled to the Electromigration Monitoring system in accordance with an aspect of the exemplary embodiment; and

FIG. 10 is a schematic block diagram of a general-purpose computer suitable for practicing the present invention exemplary embodiments.

### DETAILED DESCRIPTION

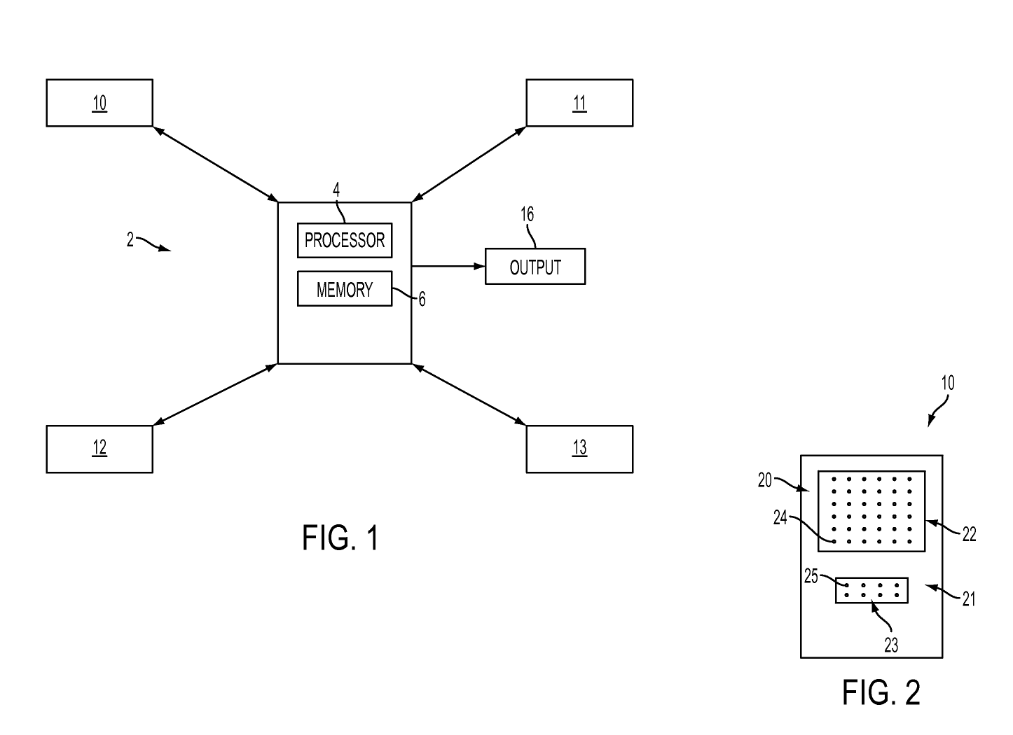

A remote electromigration monitoring system in accordance with an exemplary embodiment is indicated generally at 2 in FIG. 1. Remote electromigration monitoring system 2 includes a processor 4 and a memory 6. In the exemplary embodiment shown, remote electromigration monitoring system 2 is operatively connected with a plurality of computer systems 10-13 and an output device 16. Output device 16 may provide a signal indicating an issue with one or more computer systems 10-13. For example, as will be detailed more fully below, remote electromigration monitoring system 2 may provide an output indicating that an electronic chip in one or more computer systems 10-13 is nearing an electromigration operational life.

Reference will now be made to FIG. 2 in describing computer system 10 with an understanding that computer systems 11-13 may include similar structure. Computer system 10 includes a plurality of electronic chips, two of which are indicated at 20 and 21. Electronic chip 20 takes the form of a processor chip 22 and electronic chip 21 takes the form of a memory chip 23. Computer system 10 may include additional electronic chips, e.g., processor chips and memory chips as well as other types of electronic chips (not shown). Processor chip 22 includes a plurality of temperature sensors, one of which is indicated at 24. Similarly, memory chip 23 includes a plurality of temperature sensors, one of which is indicated at 25. Generally, processor chip 22 will include a greater number of temperature sensors than memory chip 23.

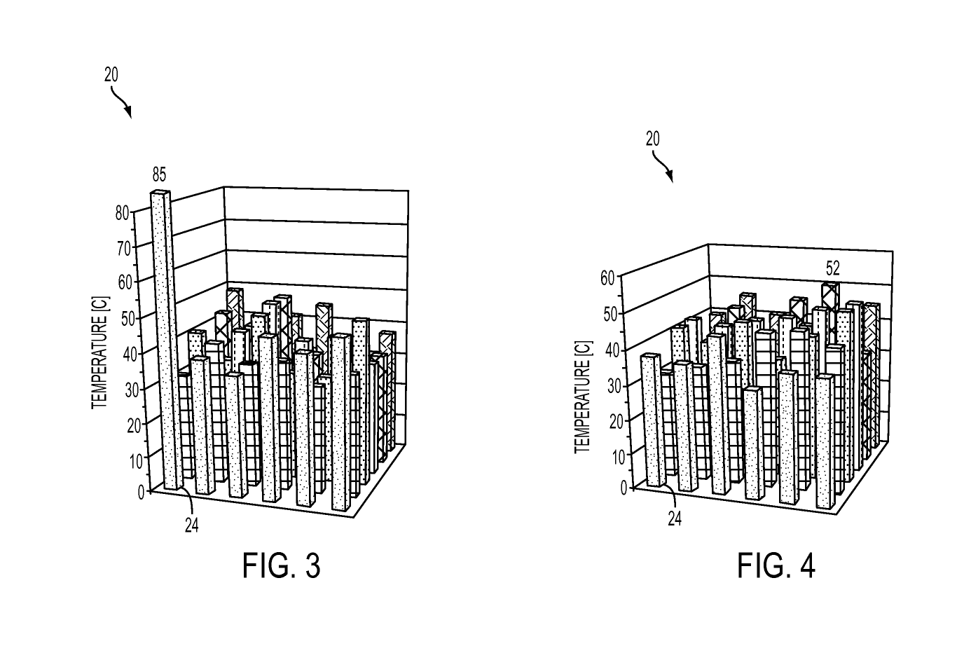

As shown in FIG. 3, temperature sensors 24 provide a temperature profile for processor chip 22. Temperature sensors 24 are typically sampled at a rate of about 32 msec. Temperatures are averaged over a reporting period and sent to remote electromigration monitoring system 2. The reporting period may vary and could represent an hour, a day or a week. The averaged temperatures provide a snap shot of the temperature profile of processor chip 22. In the example of FIG. 3, one of the temperature sensors 24 reported a temperature of 85° C. for a portion of processor chip 22. 85° C. represents a high temperature for processor chip 22 during the reporting period. In accordance with an aspect of the exemplary, the high temperature is set as the temperature for processor chip 22 for purposes of electromigration monitoring as will become more fully evident below. Remote electromigration monitoring system 2 determines an Electromigration Life Consumed (EMLC) value based on the high temperature value and Power On Hours (POH) for processor chip 22 for the reporting period.

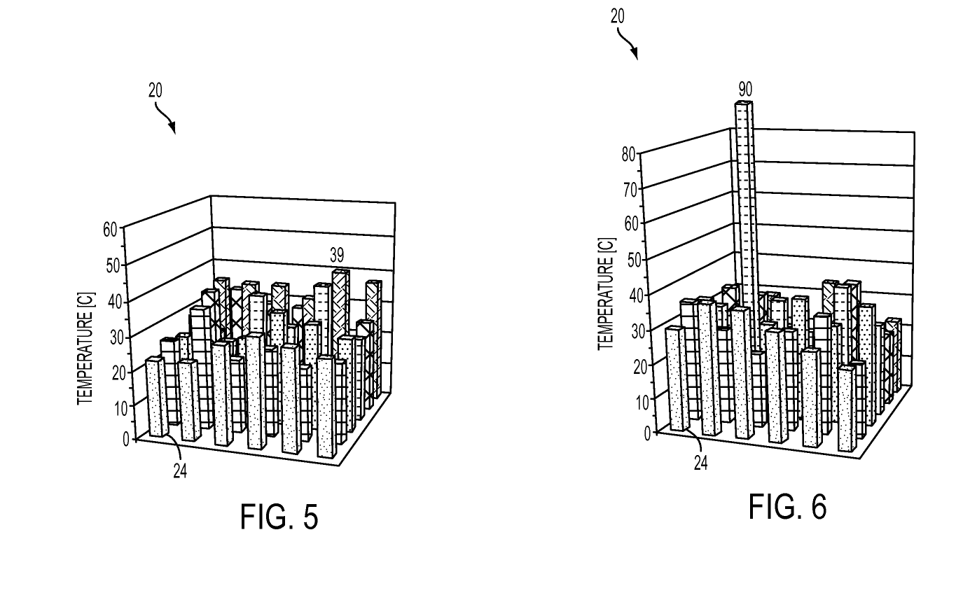

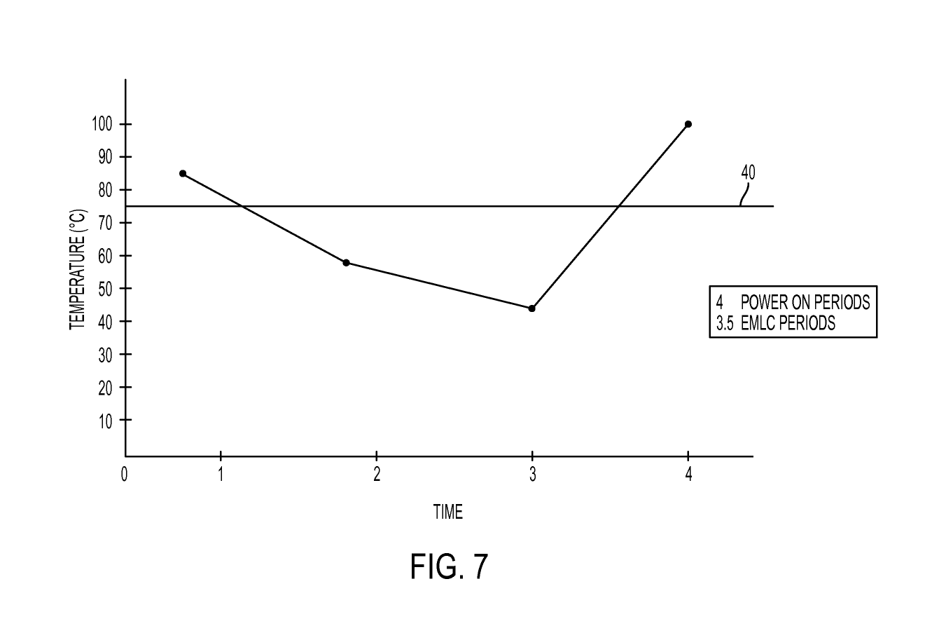

FIG. 4 illustrates another of temperature sensors 24 reporting a high temperature of 52° C. for a second reporting period. FIG. 5 illustrates one of temperature sensors 24 reporting a high temperature of 39° C. for a third reporting period and FIG. 6 illustrates one of temperature sensors 24 reporting a high temperature of 90° C. for a fourth reporting period. The high temperatures for the first, second, third and fourth reporting period are illustrated graphically in FIG. 7. As shown, two high temperatures fall above a threshold value 40 and two high temperatures fall below threshold value 40. Based on the high temperatures and POH, remote electromigration monitoring system calculates an EMLC for processor chip 22 for each reporting period as well as a total EMLC over the first, second, third and fourth reporting periods. As the high temperatures fell above and below threshold value 40, remote electromigration monitoring system 2 calculated a total EMLC for the combined four periods of POH at 3.5 periods. At this point it should be understood that the 3.5 period EMLC is provided for illustrative purposes only and should not be considered as an actual calculated EMLC based on the exemplary temperatures illustrated in FIG. 4.

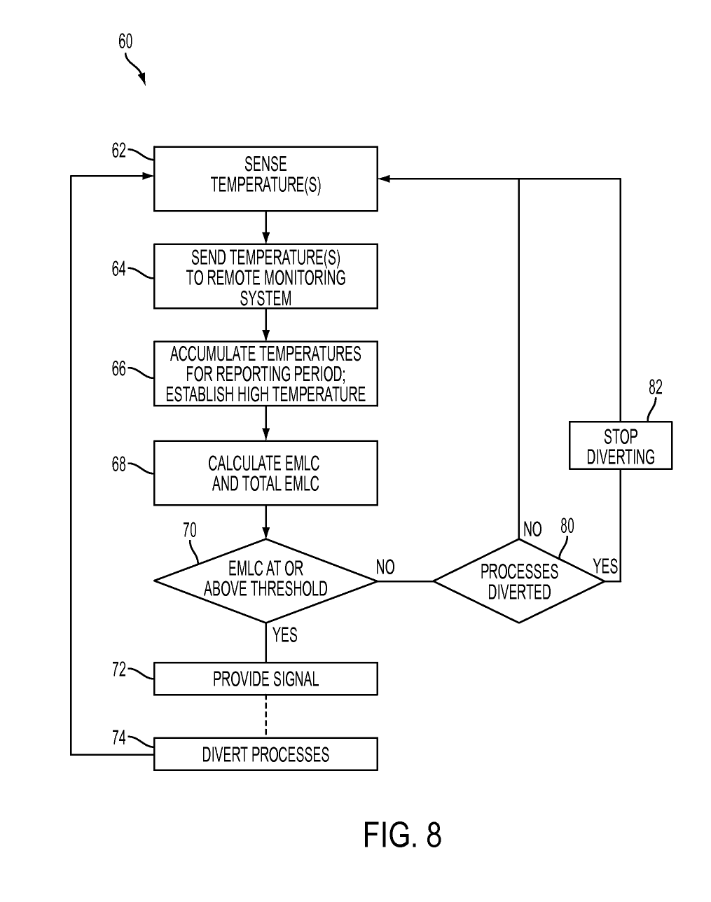

Reference will now follow to FIG. 8 in describing a method 60 of remotely monitoring electromigration in accordance with an exemplary embodiment. Temperatures are sensed at processor chip 22 at block 62. The sensed temperatures are sent to remote electromigration monitoring system 2 in block 64. Temperatures are accumulated for a reporting period, averaged, and a high temperature value for the reporting period is established in block 66. Remote electromigration monitoring system 2 calculates an EMLC for the reporting period and a total EMLC of processor chip 22 in block 68 and determines whether the EMLC is above a predetermined EMLC threshold in block 70. For example, for an electronic chip rated at 100 k POH, an exemplary EMLC threshold could be 90 k.

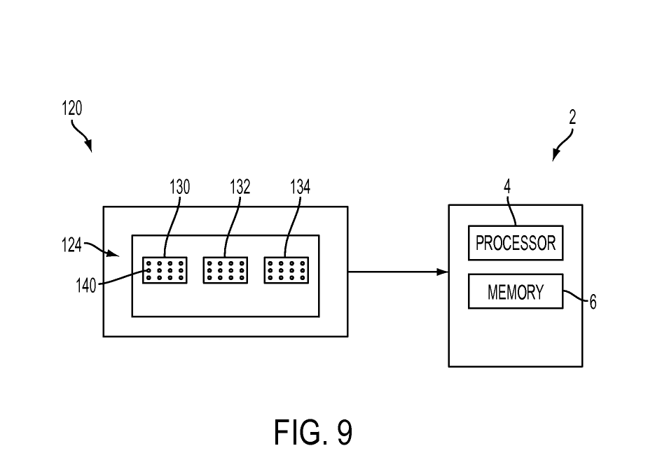

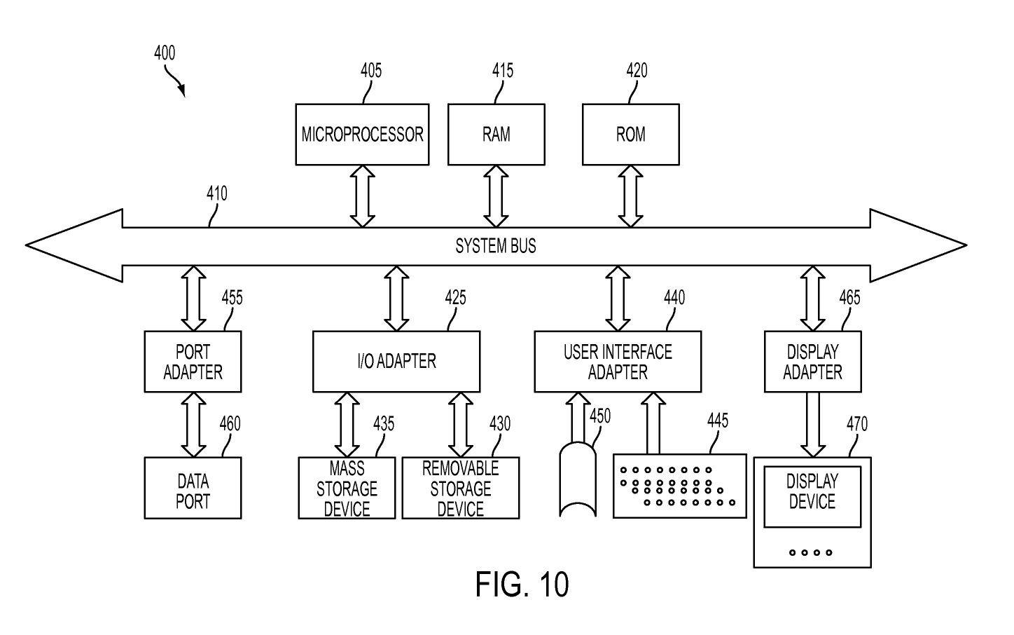

If the total EMLC in block 70 is above the EMLC threshold, remote electromigration monitoring system 2 provides a signal 72. Signal 72 may be provided on output device 16 or could take the form of diverting processing applications from processor chip 22 as shown in block 74. Of course, signal 72 may allow operators to manually divert processing applications from processor chip 22 or take steps to replace processor chip 22. If processes are diverted, monitoring continues in block 62. If the total EMLC in block 70 is below the ELMC threshold, a determination is made, in block 80, whether processes were diverted. If no processes were diverted, monitoring resumes in block 62. If processes have been diverted in block 72, the processes are directed back to processor chip 22 in block 82 and monitoring continues in block 62. In this manner, remote electromigration monitoring system 2 can take steps to reduce EMLC in chips that are at or above the predetermined EMLC threshold, or provide a signal so that operators can decide whether an electronic chip, near the end of its electromigration life, should be replaced. It should also be understood that in addition to diverting processes from processor chip 22, processes can be redistributed to other cores. As shown in FIG. 9, remote electromigration monitoring system 2 may be operatively connected to computer system 120 having a multi-core processor chip 124. Multi-core processor chip 124 includes a plurality of processor cores 130, 132 and 134 each having associated temperatures sensors such as shown at 140. Remote electromigration monitoring system 2 monitors temperatures, POH, and calculates EMLC for each core 130, 132, and 134. In the event that one or two of cores 130, 132 and 134 is at or above the EMLC threshold, processes can be diverted to others of the cores 130, 132, and 134 until the total EMLC of the affected core(s) drops below the EMLC threshold or other corrective action is taken.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one more other features, integers, steps, operations, element components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated

The capabilities of the present invention can be implemented in software, firmware, hardware or some combination thereof. As remote electromigration monitoring system 2 may be part of a larger general purpose computer system configured to calculate EMLC values for each electronic chip in each computer system. The method may be coded as a set of instructions on removable or hard media for use by the general-purpose computer. FIG. 10 is a schematic block diagram of a general-purpose computer suitable for practicing the present invention embodiments. In FIG. 10, computer system 400 has at least one microprocessor or central processing unit (CPU) 405. CPU 405 is interconnected via a system bus 410 to a random access memory (RAM) 415, a read-only memory (ROM) 420, an input/output (I/O) adapter 425 for connecting a removable data and/or program storage device 430 and a mass data and/or program storage device 435, a user interface adapter 440 for connecting a keyboard 445 and a mouse 450, a port adapter 455 for connecting a data port 460 and a display adapter 465 for connecting a display device 470.

ROM 420 contains the basic operating system for computer system 400. The operating system may alternatively reside in RAM 415 or elsewhere, as is known in the art. Examples of removable data and/or program storage device 430 include magnetic media such as floppy drives and tape drives, and optical media such as CD ROM drives. Examples of mass data and/or program storage device 435 include hard disk drives and non-volatile memory such as flash memory. In addition to keyboard 445 and mouse 450, other user input devices such as trackballs, writing tablets, pressure pads, microphones, light pens and position-sensing screen displays may be connected to user interface 440. Examples of display devices include cathode-ray tubes (CRT) and liquid crystal displays (LCD).

The flow diagrams depicted herein are just one example. There may be many variations to this diagram or the steps (or operations) described therein without departing from the spirit of the invention. For instance, the steps may be performed in a differing order or steps may be added, deleted or modified. All of these variations are considered a part of the claimed invention.

At this point it should be understood that the exemplary embodiments provide a system for remotely monitoring electromigration in electronic chips. Further, the remote electromigration monitoring system polls multiple temperature sensors on each electronic chips to get a more accurate assessment of operating conditions. Further, the remote electromigration monitoring system is configured to take steps to reduce operating temperatures of an electronic chip that has an EMLC above the EMLC threshold. In this manner, remote electromigration monitoring system may renew or elongate electromigration life of an electronic chip(s) that is above the EMLC threshold.

While the preferred embodiment to the invention had been described, it will be understood that those skilled in the art, both now and in the future, may make various improvements and enhancements which fall within the scope of the claims which follow. These claims should be construed to maintain the proper protection for the invention first described.

What is claimed is:

1. A method of remotely monitoring electromigration in an electronic chip, the method comprising:
sensing, at a first location, at least one temperature value of the electronic chip;
sending the at least one temperature value to a remote monitoring system;
accumulating a plurality of temperature values of the electronic chip at the monitoring system during a reporting period;
calculating an Electromigration Life Consumed (EMLC) value of the electronic chip for the reporting period based on the plurality of temperature values;
determining whether the EMLC of the electronic chip is above a predetermined EMLC threshold; and
providing a signal when the EMLC of the electronic chip is above the predetermined threshold.

2. The method of claim 1, wherein sensing the at least one temperature value of the electronic chip includes sensing a plurality of temperature values, each of the plurality of temperature values being at a different position on the chip.

3. The method of claim 2, further comprising:
determining a high temperature value of the plurality of temperature values;
setting the at least one temperature value of the electronic chip at the high temperature value.

4. The method of claim 2, further comprising: sending the plurality of temperature values to the remote monitoring system to develop a temperature profile of the electronic chip.

5. The method of claim 1, further comprising:
determining when the at least one temperature value is below the predetermined threshold; and
adjusting the EMLC based on an amount of time the at least one temperature value is below the predetermined threshold.

6. The method of claim 1, wherein providing the signal when the EMLC of the electronic chip is above the predetermined threshold includes diverting processing operations from the electronic chip.

7. The method of claim 1, wherein sensing at least one temperature value of an electronic chip includes sensing a temperature value for each of a plurality of cores of the electronic chip.

8. The method of claim 7, wherein providing the signal when the EMLC of the electronic chip is above the predetermined threshold includes providing a signal when the EMLC of one of the plurality of cores is above the predetermined threshold, and diverting processing operations from the one of the plurality of cores to others of the plurality of cores.

9. A computer program product comprising:
a computer useable medium including a computer readable program, wherein the computer readable program, when executed on a computer, causes the computer to:
sense, at a first location, at least one temperature value of the electronic chip;
send the at least one temperature value to a remote monitoring system;
accumulate a plurality of temperature values of the electronic chip at the monitoring system during a reporting period;
calculate an Electromigration Life Consumed (EMLC) value of the electronic chip for the reporting period based on the plurality of temperature values;
determine whether the EMLC of the electronic chip is above a predetermined threshold; and
provide a signal when the EMLC of the electronic chip is above the predetermined threshold.

10. The computer program product of claim 9, wherein the computer readable program, when executed on a computer causes, the computer to: sense a plurality of temperature values, each of the plurality of temperature values being at a different position on the chip.

11. The computer program product of claim 10, wherein the computer readable program, when executed on a computer, causes the computer to:
determine a high temperature value of the plurality of temperature values;
set the at least one temperature value of the electronic chip at the high temperature value.

12. The computer program product of claim 9, wherein the computer readable program, when executed on a computer, causes the computer to:
determine when the at least one temperature value is below the predetermined threshold; and
adjust the EMLC based on an amount of time the at least one temperature value is below the predetermined threshold.

13. The computer program product of claim 9, wherein the computer readable program, when executed on a computer, causes the computer to: divert processing operations from the electronic chip when the EMLC is above the predetermined threshold.
